(19) United States
(12) Patent Application Publication
(10) Pub. No.: US 2024/0032816 A1
(43) Pub. Date: Feb. 1, 2024

(54) METHOD AND APPARATUS FOR REAL TIME RESPIRATORY MONITORING USING EMBEDDED FIBER BRAGG GRATINGS

(71) Applicant: EmpNia Inc., Edina, MN (US)

(72) Inventor: Manojeet Bhattacharya, Edina, MN (US)

(21) Appl. No.: 18/006,144

(22) PCT Filed: Jul. 21, 2021
(86) PCT No.: PCT/US2021/042641

§ 371 Date: Jan. 19, 2023

Related U.S. Application Data
(60) Provisional application No. 63/054,874, filed on Jul. 22, 2020.

(51) Int. Cl. A61B 5/08; A61B 5/0205; A61B 5/00

(52) U.S. Cl. CPC A61B 5/0816; A61B 5/0803; A61B 5/0205; A61B 5/7275; A61B 5/742; A61B 5/746; A61B 5/6801; A61B 5/0022; A61B 2560/04; A61B 2562/046

(57) ABSTRACT

A wearable device for monitoring respiratory function includes a front portion having embedded fiber Bragg gratings (FBGs). The device includes at least one light emitter, each light emitter configured to pulse light waves through a corresponding FBGs. The device further includes at least one light sensor configured to receive pulsed light waves. A processor receives from the light sensors peak wavelengths reflected by the at least one FBG and detects effective shifts of the Bragg wavelengths of the at least one FBG caused by body deformation over a period of time to establish a baseline respiratory pattern, the device may compare the baseline respiratory pattern with profiled respiratory patterns to determine whether the baseline respiratory pattern is indicative of a potential disease state and provide an alert of the potential disease state.

### RELATED APPLICATION

This application is claims the benefit of U.S. Provisional Application No. 63/054,874, filed on Jul. 22, 2020. The entire teachings of the above application are incorporated herein by reference.

### BACKGROUND

Monitoring the respiratory state of a person is an important aspect of health monitoring in many circumstances. These include post-operative patients, those who suffer from chronic cardio-pulmonary diseases, and those with respiratory infections such as those infected by the COVID-19. The respiratory rate of an individual can be measured as the number of breaths a person takes within a certain amount of time, such as breaths per minute. In addition, respiratory patterns that may consist of the amplitude and duration of inhalation and exhalation contain clinical indications of the normal and abnormal respiratory state. Fluctuations in the respiratory patterns induced by the cardiac cycle, called the cardiogenic oscillations, may be indicators of cardiac conditions such as heart failure. The tidal volume defines the volume of air that moves through the lungs during a breath, and the product of the tidal volume and the respiratory rate defines the minute ventilation, an important measure of respiratory health. In a hospital setting, a clinician may use respiratory rate, minute ventilation, as well as respiratory pattern measurements to determine whether a patient is experiencing respiratory distress and/or dysfunction. Further, respiratory rate and pattern measurements may also be used in sports medicine for evidence based in fitness/endurance assessments of athletes as well as general population.

### SUMMARY

Embodiments consistent with principles of the present invention include a method and system for monitoring an individual's respiratory function to detecting a baseline respiratory frequency and any abnormal changes in the respiratory signals that may be indicative of a disease or fitness state.

In one embodiment, a wearable device with embedded fiber Bragg gratings (FBGs) can be worn on individual's body in a manner that may detect the expansion and contraction of the body at respiratory frequencies by measuring the time dependent shifts of the Bragg wavelengths due to the induced strain on the FBGs from body deformation. By detecting effective shifts of the Bragg wavelengths of the FBGs caused by body deformation over a period of time, the device may establish a baseline respiratory pattern. In some embodiments, the wearable device may be a wearable strap wrapped lightly round the thorax or the abdomen. In other embodiments, the wearable device may be a patch with embedded FBGs attached with adhesives to the thorax or the abdomen. Using the FBGs, the device may compare the baseline respiratory pattern with profiled respiratory patterns to determine whether the baseline respiratory pattern is indicative of a potential disease state and provide an alert of the potential disease state. In yet other embodiments, the device may be a pad to be placed under an individual, or as a blanket to be placed on top of an individual such that the embedded FBGs can detect an individual's respiratory movement. Such an embodiment may be particularly useful in a hospital setting, where the device may provide the FBG data to a processor and interface to provide health care professionals with monitoring of a patient's respiratory patterns.

In other embodiments, the device may further continue to acquire wavelength data from the plurality of FBGs to detect any changes in the any abnormal changes in the respiratory signals beyond a set threshold that may be indicative of a disease state; and provide an alert of the potential disease state.

Such a device could be part of hospital based critical or inpatient care system or a home healthcare system. In another form factor such a device can be a wearable system for the general population as part of a comprehensive “wearable continuous vitals monitoring system” for the general population as part of mobile health or telemedicine. Such a device could also be used for real time continuous infant health monitoring.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a representative FBG in a fiber-core.

FIG. 2 is an embodiment of a device that may be used to monitor real time respiratory functions according to principles of the present invention.

FIG. 3 is another embodiment of a device that may be used to monitor real time respiratory functions according to principles of the present invention.

FIG. 4 is another embodiment of a device that may be used to monitor real time respiratory functions according to principles of the present invention.

FIG. 5 is another embodiment of a device that may be used to monitor real time respiratory functions according to principles of the present invention.

FIG. 6 is a flowchart illustrating a method of monitoring real time respiratory functions according to principles of the present invention.

FIG. 7 is an embodiment of a system that may be used to monitor real time respiratory functions according to principles of the present invention.

### DETAILED DESCRIPTION

A description of example embodiments follows.

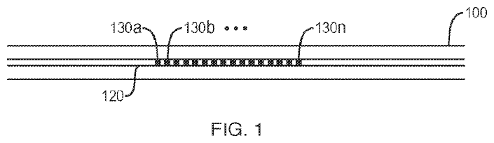

As illustrated in FIG. 1, a fiber Bragg grating (FBG) 100 is a small length of optical fiber 120 that comprises a plurality of reflection points 130a-n that create a periodic variation of refractive index. The FBG reflects a unique wavelength (λB), centered around a bandwidth, ΔλB. The periodicity Λ of the grating is related to the Bragg wavelength λB.

1B=2·n_{eff}Λ (1)

n_{eff} is the effective refractive index of the single-mode photosensitive fiber. As the fiber is stretched and grating parameter Λ increases by δΛ while effective refractive index n_{eff} decreases by δn_{eff}. The Bragg wavelength λB shifts by

δ1B=2{n_{eff}·δΛ+Λ·δn_{eff}} (1a)

By embedding one or more optical fibers with one or more FBG in wearable materials that can be wrapped over parts of anatomically relevant parts of the human body, a wearable device can be used to sense the deformation of that part resulting from physiological processes such as breathing. In certain embodiments consistent with principles of the invention, the deformation data may be used to measure and establish respiratory and cardiac patterns in a body.

Before one can use the embedded FBG as a strain gauge, the FBG's response function and linearity should be characterized as a function of load. To characterize the FBG's response function and linearity, an electrical strain gauge may be used to calibrate the FBG such that the applied tensile loading approximates readings of the displacement of the body within the Cartesian coordinate system for a three-dimensional object. For the FBG to perform as a reliable strain gauge, the change in the reflection wavelength of the FBG as it gets stretched under tensile load must linearly track the electrical strain gauge data. Once calibrated, the response of an FBG may be reliably used as an embedded strain gauge for detecting object surface deformation. Within reasonable limits on the elasticity of the gauge, it may also be used for detecting the degree to which the object surface has been displaced. Based on a calibration curve comparing pressure against strain or wavelength, along with the strain data from the sensors, one can detect the degree of displacement. In other cases, the calibration curves may be derived from comparing reflected Bragg wavelengths to secondary respiratory measurements that can include physical or image based measurements.

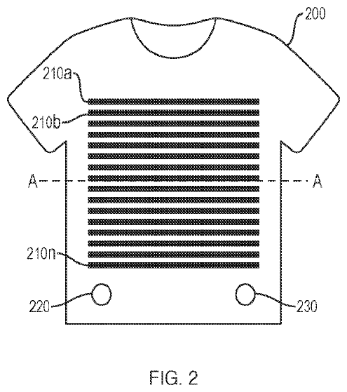

FIG. 2 is an embodiment if a garment 200 worn by a patient and used to monitor respiratory activity in accordance with principles of the present invention. In the garment 200, a plurality of FBG fibers 210a-n are embedded laterally along the garment, running in a direction parallel to a scanning plane A. Garment 200 may have an input 220 for a laser or light source that is transmitted through the FBG fibers 210a-n. Each FBG 210a-n in connected to a light sensor (not shown) that receives pulsed light waves from the light sources. In addition, the garment 200 may also include an output 230, where the light sensors may provide data concerning the light transmission through each of the FBGs 210a-n to an external processor that can identify shifts in the refractive index of the FBGs 210a-n, suggesting deformations in the surface of the object within the garment. In other embodiments, the processor may be internal to the garment, and transmit data via a wireless transmission, such as WiFi or Bluetooth. The multiple FBGs 210a-n can help identify where in the cross-sectional scanning plane there may be specific movement, as each provides a different longitudinal marker along a cartesian coordinate system. The FBGs may enable the system to measure displacement over a period of time to establish respiratory patterns consisting of the amplitude and duration of inhalation and exhalation, as well as respiratory rates. Given the low attenuation properties of this garment and that the fiber optic sensors do not create electromagnetic interference with other sensor systems, including imaging systems, it may be used in connection with other physiological monitoring systems.

The change in wavelength measured over time for a free breathing patient wearing such a garment represents the patient specific respiratory signal. The respiratory signal may be compared to known respiratory patterns indicative of disease states, or monitored to detect for changes in respiratory patterns that may indicate potential disease states or the onset of such a state.

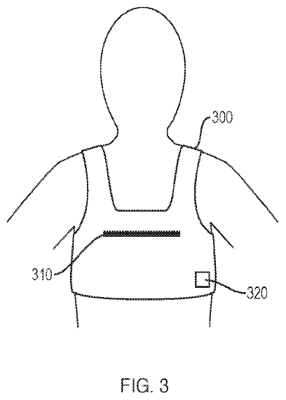

FIG. 3 is another embodiment of a wearable strap 300 that may be used to monitor respiratory activity according to principles of the present invention. In this garment, at least one FBG fiber 310 is embedded longitudinally along the strap. In addition, the garment 300 may also include a processor 320, that controls the light emitters (not shown) through the FBG and sensor (not shown) to receive may provide data concerning the light transmission through the FBG 310. The processor 320 may send the sensor data to remote processor. In some embodiments, the processor 320 may send the data through a wired connection. In other embodiments, the processor may transmit data via a wireless transmission, such as WiFi or Bluetooth.

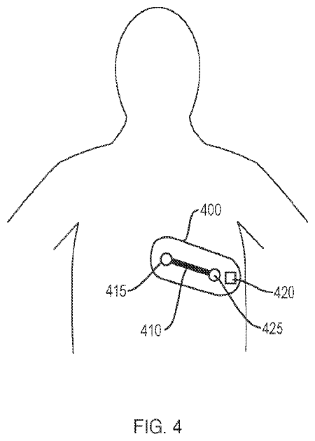

FIG. 4 is yet another embodiment consistent with principles of the present invention to monitor respiratory activity. A patch 400 that may be attached to a patient using adhesives. In this patch, at least one FBG fiber 410 is embedded longitudinally along the patch. In addition, the garment 300 may also include a processor 420, that controls the light emitter 425 through the FBG and sensor 415 to receive may provide data concerning the light transmission through the FBG 410. The processor 420 may send the sensor data to remote processor. In some embodiments, the processor 420 may send the data through a wired connection. In other embodiments, the processor may transmit data via a wireless transmission, such as WiFi or Bluetooth.

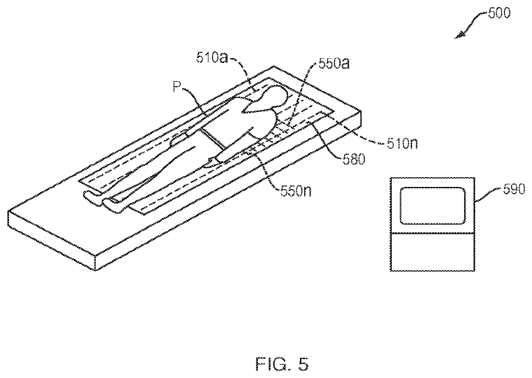

In yet other embodiments, as illustrated in FIG. 5, a patient monitoring system 500 may include padding 580 that has fibers containing the FBGs embedded similar to the garments illustrated in FIGS. 2, 3, and 4. As shown in FIG. 5, a plurality of FBG fibers 510a-n are embedded longitudinally along the padding 580, and another plurality of FBG fibers 550a-n are embedded latitudinally along the padding. In alternate embodiments consistent with the teachings herein, the padding 580 may have FBGs embedded in other configurations to provide data relating to movement or displacement of a body B on the padding. Such fibers can also be embedded directly in the patient handling systems (patient beds) of medical imaging and radiation therapy devices. As with the garments shown in FIGS. 2, 3, and 4, the padding may include an output (not shown), where light sensors may provide data to an external processor and interface 590. In some embodiments, the interface may be a mobile device or tablet. The FBGs may be used for monitoring the respiratory patterns of the body P in contact with the padding 580. The interface 590 may provide an easily accessible view of a patient's vital signs, and other physiological information. In some embodiments, the padding 580 may be a blanket that rests on top of the patient.

In embodiments of the garment with embedded FBGs for real time measurement of the deformation of the patient body under respiration, one may embed a number of FBGs using a predetermined coordinate system, such as a cartesian coordinate system or polar coordinate system. Additionally, the predetermined coordinate system may be determined in such a way as to balance competing interests of maximizing the fidelity of the measured deformation map while also using the least number of embedded FBGs. This could mean that the embedded FBGs are aligned along a coordinate system with respect to the patient body or in other cases they could be located for a pseudorandom sampling of the patient body. In some embodiments, this could mean that the FBGs could be distributed such that a concentration of embedded FBGs are aligned in a more dense distribution in one region, and loosely distribute in others. Depending on the nature of the garment, the distribution of FBGs within the garment may vary, as a belt or shirt may have a different, more contoured fit around a body than a blanket. Additionally, multiple FBGs can be inscribed inside a single mode optical fiber, and as long as they are separated by an predetermined optimal distance from each other and that each of these FBGs have a unique and distinct Bragg wavelength, a single such optical fiber can be used to measure the strain along its length using a single broadband light source and a single wavelength multiplex detection system. Such a system has distinct advantages over an electrical strain gauge-based system as in the latter case each strain gauge needs is own electrical connection.

Once collected, data may be used to create algorithms designed to spot physiological changes that happen to the wearer and indicate sickness is on the way. These changes involve certain changes in respiratory patterns or changes within certain thresholds that might be indicative of particular conditions. As additional data concerning users is gathered, the system can employ machine learning and predictive models to identify the onset of those conditions in patients or users. The system can then warn the wearer with an alert that they may be developing an illness or particular symptoms requiring treatment. In another use case these observed changes can be used to adapt and optimize training for professional athletes and exercise regimens of general population using connected exercise equipment. As an example, it may be used to monitor respiratory activity during a training session to ensure the wearer does not exceed certain respiratory thresholds that may be unsafe. These thresholds may be set based on an individual's personal health history, or based on collective respiratory profiles. In the fitness/endurance monitoring case, the detected respiratory patterns may also be used to optimize and individualize training/exercise regimes based on baseline data and thresholds.

By embedding one or more optical fibers with one or more FBGs in wearable materials that can be wrapped over parts of anatomically relevant parts of the human body, the one or more FBGs can be configured to sense the motion resulting from physiological processes such as breathing, heart beats, blood pressure, and blood flow. In some embodiments, the respiratory data may be used with other monitored physiological data (whether from FBGs or other monitoring means) to provide a more comprehensive view of the monitored individual, and employ better predictive models to identify the onset of conditions in patients or users.

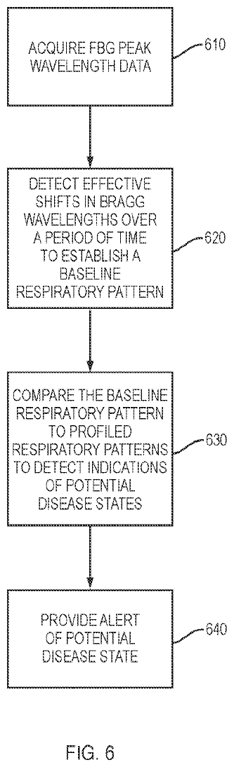

FIG. 6 is a flowchart illustrating a method of monitoring real time respiratory functions according to principles of the present invention. At step 610, a device having embedded FBGs in contact with the body of a patient may acquire FBG peak wavelength data. At step 620, the acquired data may be measured over a period of time and used to detect effective shifts in Bragg wavelengths due to body deformation caused by respiratory activity. This data may be used to establish a baseline respiratory pattern. At step 630, the baseline respiratory pattern may be compared to profiled respiratory patterns stored in a database in order to detect any indications of potential disease states. These diseases may include respiratory patters consistent with symptoms of a coronavirus, severe acute respiratory syndrome (SARS), or acute asthma. If the baseline respiratory pattern is consistent with a potential disease state, at Step 640 the system can provide an alert of that potential disease state to the patient, a caregiver, or health care personnel. This alert may be provided in an interface such as a dedicated monitor, an alert to a handheld device, or in some embodiments, on an interface on the wearable device having the embedded FBGs.

In other embodiments, if the system does not detect any indications of potential disease states, it may continue to monitor the respiratory patterns of the patient. If the respiratory patterns change above certain thresholds (based on either respiratory rates, amplitudes or duration), the thresholds either manually set by the patient or caregiver, or learned by the system through training algorithms, an alert can be provided to an interface. In some embodiments consistent with principles of the invention, baseline respiratory patterns may be collected at a database and processed through a training algorithm to help the system identify profiled respiratory patterns or respiratory thresholds.

In yet other embodiments, if the system does detect an indication of potential disease states, it may continue to monitor the respiratory patterns of the patient to detect improvements in their respiratory state. If such a change were to occur, the system could then provide an alert indicating that improvement.

While the prior art teaches different means of measuring respiratory rate, the use of embedded FBGs in a measurement device provides a level of sensitivity and precision of monitoring not capable in those prior art systems. The examples below illustrate the various applications of the monitoring capabilities of embedded FBGs.

### Non-Invasive Minute Ventilation Monitoring

As one example, respiratory rate despite being a key indicator of human health does not provide sufficient information on the pulmonary state of a patient as it does not contain any information on respiratory volume changes, a key component of what is known as the “minute ventilation” defined as the product of the respiratory rate and tidal volume. Minute ventilation has shown to be an early indicator of pulmonary distress compared to pulse oximetry measurements. Currently available non-invasive minute ventilation methods include spirometry that is prone to errors due to significant patient training and compliance required, and end-tidal CO2 measurements that are used only for intubated patients. Recently there has been interest in impedance pneumography via the measurements of transthoracic impedance as a tool for non-invasive measurements of tidal volumes and in turn minute ventilation. This method requires measuring small changes in impedance under respiration and is dependent on placement of electrodes.

Wearable devices with embedded FBGs on the other hand, can measure minute changes in the strain that are more than two orders of magnitude smaller than those induced under respiration, as a consequence such devices can perform very accurate non-invasive and continuous measurements of tidal volumes and minute ventilation in multitude of circumstances, from hospital and home healthcare to sports and fitness training.

### Monitoring Cardiogenic Oscillations for Monitoring Heart Failure

Cardiogenic oscillations in the respiratory waveform induced by the variations of pulmonary blood volume with correspondence to the cardiac cycle of a patient has been shown to be an indicator of Heart Failure (HF). Heart Failure and in particular, Acute Decompensated Heart Failure (ADHF) due to many underlying conditions is serious condition that often results in respiratory distress and hospitalization. Continuous cardiac function and respiratory monitoring of HF patients is highly desirable but currently all available solutions for such monitoring such as Pulmonary Artery Catheterization (PAC) are invasive procedures and can only be performed in the clinical setting under physician supervision.

Measurements of Cardiogenic Oscillations of Respiratory Waveforms (small waveforms superimposed on pressure and flow signals) is known to be a promising method for monitoring respiratory system mechanics, cardiac function, and heart failure. A wearable respiratory monitoring system based on embedded FBG strain sensing has the sensitivity for measuring these oscillations and can be a promising device for non-invasive cardiac monitoring and HF.

### Infant Physiologic Monitoring Systems

Physiologic monitoring of infants while sleeping or otherwise unsupervised and alerts based on onsets of adverse events is an important area in of health monitoring that has seen growth with the advent of new technology for non-invasive and remote monitoring. One particular method for infant physiologic monitoring has been the use of pulse oximetry. However, as described above, that change in pulse oximetry is known to be a late indicator of respiratory distress. As an alternative, a wearable non-invasive respiratory monitoring system based on embedded FBG strain measurement system can be the true real time infant physiologic monitoring system that is currently not available in the market.

### Embedded Physiological Monitoring System

Sleep monitoring in general, and physiologic monitoring of humans while sleeping in particular, are important parts of health monitoring that can provide insights to human health and can be an important tool for management of chronic conditions such as Asthma and Apnea. However, there has been no commercially available non-invasive physiologic monitoring system available for general population. Once available, such systems and the data that they can acquire and the machine learning that they can enable will provide new insights into health conditions that have not been available so far. A physiologic monitoring system based on dynamic strain sensing using embedded FBGs in wearable straps or in the bedding can provide an easy to use non-invasive physiologic monitoring system that can provide such data even during sleep and can help usher a new era of proactive healthcare.

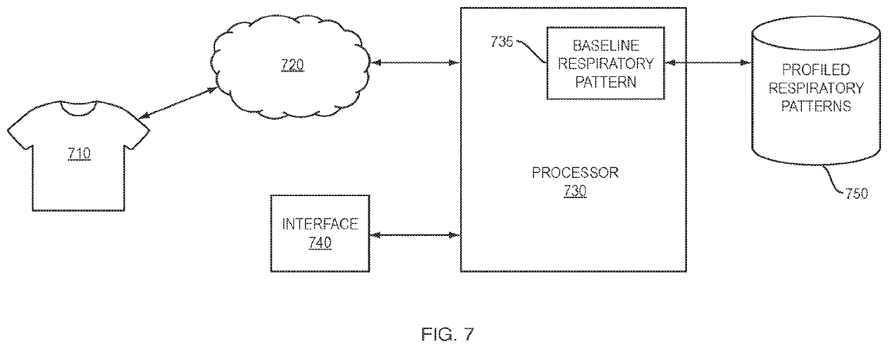

FIG. 7 is an embodiment of a system 700 that may be used to monitor real time respiratory functions consistent with principles of the present invention. An individual may wear the respiratory monitoring device 710. Such device 710 may be the garment 200 of FIG. 2, the strap of FIG. 3, the patch of FIG. 4, the padding of FIG. 5, or some other device consistent with principles of the invention. The device 710 obtains data from the light sensors and sends them through a network 720 to a processor 730 that uses the data to detect effective shifts of the Bragg wavelengths of the at least one FBG caused by body deformation over a period of time to establish a baseline respiratory pattern 735. The processor 730 is configured to compare the baseline respiratory pattern 735 with profiled respiratory patterns stored in a database 750 to determine whether the baseline respiratory pattern is indicative of a potential disease state. If a potential disease state is detected, the processor 730 may provide an alert of the potential disease state.

In some embodiments, the processor 730 may be configured to monitor the baseline respiratory pattern for any significant changes in the respiratory patterns, including changes in a person's minute ventilation as discussed above. By detecting any threshold changes, or changes in pattern, the system can provide an alert of the onset of change in condition, or use predictive algorithms to warn of a potential change in condition to allow the user or medical personnel to take action prior to the onset.

In some embodiments, the processor 730 may be located locally on the device 710. In other embodiments, the processor 730 may be located on a remote general purpose computer or cloud based processor. The interface may include a display on a computer, a display on a handheld device, a display on the wearable device 710, or may take the form of an audio alert or text message on a mobile phone. Consistent with other embodiments, similar system may be used to monitor real time respiratory patterns to optimize and individualize training/exercise regimes based on baseline data and thresholds. Such embodiments may present the respiratory patterns to the user over the interface 740, and provide alerts related to changes in those patterns.

While example embodiments have been particularly shown and described, it will be understood by those skilled in the art that various changes in form and details may be made therein without departing from the scope encompassed by the appended claims.

It should be understood that the example embodiments described above may be implemented in many different ways. In some instances, the various methods and machines described herein may each be implemented by a physical, virtual or hybrid general purpose computer having a central processor, memory, disk or other mass storage, communication interface(s), input/output (I/O) device(s), and other peripherals. The general purpose computer is transformed into the machines that execute the methods described above, for example, by loading software instructions into a data processor, and then causing execution of the instructions to carry out the functions described, herein.

As is known in the art, such a computer may contain a system bus, where a bus is a set of hardware lines used for data transfer among the components of a computer or processing system. The bus or busses are essentially shared conduit(s) that connect different elements of the computer system, e.g., processor, disk storage, memory, input/output ports, network ports, etcetera, which enables the transfer of information between the elements. One or more central processor units are attached to the system bus and provide for the execution of computer instructions. Also attached to system bus are typically I/O device interfaces for connecting various input and output devices, e.g., keyboard, mouse, displays, printers, speakers, etcetera, to the computer. Network interface(s) allow the computer to connect to various other devices attached to a network. Memory provides volatile storage for computer software instructions and data used to implement an embodiment. Disk or other mass storage provides non-volatile storage for computer software instructions and data used to implement, for example, the various procedures described herein.

Embodiments may therefore typically be implemented in hardware, firmware, software, or any combination thereof.

In certain embodiments, the procedures, devices, and processes described herein constitute a computer program product, including a non-transitory computer-readable medium, e.g., a removable storage medium such as one or more DVD-ROM's, CD-ROM's, diskettes, tapes, etcetera, that provides at least a portion of the software instructions for the system. Such a computer program product can be installed by any suitable software installation procedure, as is well known in the art. In another embodiment, at least a portion of the software instructions may also be downloaded over a cable, communication and/or wireless connection.

Further, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etcetera.

It also should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

While this invention has been particularly shown and described with references to example embodiments thereof, it will be understood by those skilled in the art that various changes in form and details may be made therein without departing from the scope of the invention encompassed by the appended claims.

What is claimed is:

1. A method for monitoring respiratory rate of a body, the method comprising:
acquiring peak wavelength data from a plurality of fiber Bragg gratings (FBGs) disposed in contact with a body;
determining effective shifts of Bragg wavelengths of the at least one FBG due to axial strain on the FBG caused by body deformation over a period of time to establish a baseline respiratory pattern;
comparing the baseline respiratory pattern with profiled respiratory patterns to determine whether the baseline respiratory pattern is indicative of a disease state;
providing an alert of the disease state.

2. The method of claim 1 further comprising:
evaluating additional physiological measures associated with the disease state to determine whether the baseline respiratory pattern is indicative of the disease state.

3. The method of claim 1 further comprising:
continuing to acquire wavelength data from the plurality of FBGs to detect any changes in the respiratory signals beyond a set threshold that may be indicative of a potential disease state; and
providing an alert of the potential disease state.

4. The method of claim 3 further comprising:
evaluating additional physiological measures associated with the potential disease state to determine whether the baseline respiratory pattern is indicative of the potential disease state.

5. The method of claim 3 wherein the abnormal changes in the respiratory signals include cardiogenic oscillations indicative of heart failure.

6. The method of claim 1 wherein the baseline respiratory patterns and the profiled respiratory patterns are minute ventilation patterns.

7. The method of claim 1 further comprising:
receiving an indication that the baseline respiratory pattern is associated with a diseased state;
updating the profiled respiratory patterns with the baseline respiratory pattern for identifying the diseased state.

8. The method of claim 6 wherein updating the profiled respiratory patterns includes providing physiological status information for identifying the diseased state.

9. A wearable device for monitoring respiratory rate of a body, the method comprising:
a front portion, made of a compression material and having at least one fiber Bragg grating (FBG), the front portion disposed in contact with a body;
at least one light emitter, each light emitter configured to pulse light waves through a corresponding FBGs;
at least one light sensor, each light sensor attached to a corresponding FBG and configured to receive pulsed light waves;
a processor configured to
i. receive from the light sensors peak wavelengths reflected by the at least one FBG;
ii. determine effective shifts of Bragg wavelengths of the at least one FBG; due to axial strain on the FBG caused by body deformation over a period of time to establish a baseline respiratory pattern;
iii. compare the baseline respiratory pattern with profiled respiratory patterns to determine whether the baseline respiratory pattern is indicative of a disease state; and
iv. provide an alert of the disease state.

10. The device of claim 9 wherein the processor is further configured to evaluate additional physiological measures associated with the disease state to determine whether the baseline respiratory pattern is indicative of the disease state.

11. The device of claim 9 wherein the processor is further configured to
i. continue to acquire wavelength data from the plurality of FBGs to detect any changes in the respiratory signals beyond a set threshold that may be indicative of a potential disease state; and
ii. provide an alert of the potential disease state.

12. The device of claim 11 wherein the processor is further configured to:
evaluate additional physiological measures associated with the potential disease state to determine whether the baseline respiratory pattern is indicative of the potential disease state.

13. The device of claim 11 wherein the abnormal changes in the respiratory signals include cardiogenic oscillations indicative of heart failure.

14. The device of claim 9 wherein the baseline respiratory patterns and the profiled respiratory patterns are minute ventilation patterns.

15. The device of claim 9 further wherein the processor is further configured to:
receive an indication that the baseline respiratory pattern is associated with a diseased state;
update the profiled respiratory patterns with the baseline respiratory pattern for identifying the diseased state.

16. The device of claim 15 wherein the processor is further configured to update the profiled respiratory patterns by providing physiological status information for identifying the diseased state.

17. The device of claim 9 wherein the front portion fits around the body.

18. A system for monitoring the physiological state of a user, the system comprising:
a wearable device including:
a. a front portion, made of a compression material and having at least one fiber Bragg grating (FBG), the front portion disposed in contact with a body;
b. at least one light emitter, each light emitter configured to pulse light waves through a corresponding FBGs;
c. at least one light sensor, each light sensor attached to a corresponding FBG and configured to receive pulsed light waves;
a database of profiled respiratory patterns indicative of potential disease states;
a processor configured to:
a. receive from the wearable device, peak wavelengths reflected by the at least one FBG;
b. determine effective shifts of Bragg wavelengths of the at least one FBG; due to axial strain on the FBG caused by body deformation over a period of time to establish a baseline respiratory pattern;
c. compare the baseline respiratory pattern with profiled respiratory patterns from the database to detect any changes in the respiratory signals beyond a set threshold that may be indicative of a potential disease state; and
d. provide an alert of the potential disease state; and
a display for providing information regarding the baseline respiratory pattern and the alert of the potential disease state.

19. The system of claim 18 wherein the database is in networked communications with the processor.

20. The system of claim 18 wherein the display is further configured to provide additional physiological status information of the body.
